(19) United States
(12) Patent Application Publication
(10) Pub. No.: US 2024/0382556 A1
(43) Pub. Date: Nov. 21, 2024

(54) METHODS AND COMPOSITIONS FOR THE TREATMENT OF ALCOHOL DEPENDENCE

(71) Applicant: Duke University, Durham, NC (US)

(72) Inventor: Cynthia Kuhn, Durham, NC (US)

(21) Appl. No.: 18/666,345

(22) Filed: May 16, 2024

Related U.S. Application Data
(60) Provisional application No. 63/466,838, filed on May 16, 2023.

(51) Int. Cl. A61K 38/07; A61P 25/32

(52) U.S. Cl. CPC A61K 38/07; A61P 25/32

(57) ABSTRACT

The present invention relates to methods of treating or preventing alcohol dependence, enhancing the treatment of alcohol dependence, treating alcohol withdrawal, reducing alcohol consumption, alleviating one or more alcohol withdrawal symptoms in a subject, preventing or reducing the likelihood of alcohol dependence relapse in a subject treated for alcohol dependence, preventing alcohol-related N-methyl-D-aspartate receptor (NMDA) upregulation, preventing alcohol-related NMDA receptor activity, comprising administering a therapeutically effective amount of an NMDA receptor partial agonist (e.g., rapastinel) to the subject. The present invention further relates to compositions comprising an NMDA receptor partial agonist (e.g., rapastinel) for use with the aforementioned methods.

### CROSS-REFERENCE TO RELATED APPLICATIONS

The present application claims priority to U.S. Provisional Application No. 63/466,838, filed May 16, 2023, which is incorporated herein by reference in its entirety.

### FIELD OF THE INVENTION

The present invention relates to methods of treating or preventing alcohol dependence, enhancing the treatment of alcohol dependence, treating alcohol withdrawal, reducing alcohol consumption, alleviating one or more alcohol withdrawal symptoms in a subject, preventing or reducing the likelihood of alcohol dependence relapse in a subject treated for alcohol dependence, preventing alcohol-related N-methyl-D-aspartate receptor (NMDA) upregulation, preventing alcohol-related NMDA receptor activity, comprising administering a therapeutically effective amount of an NMDA receptor partial agonist (e.g., rapastinel) to the subject. The present invention further relates to compositions comprising an NMDA receptor partial agonist (e.g., rapastinel) for use with the aforementioned methods.

### BACKGROUND OF THE INVENTION

Alcohol remains the most used of all addictive drugs. According to the National Institute on Alcohol Abuse and Alcoholism, 15 million Americans have alcohol use disorder (alcoholism/alcohol dependence). The CDC estimates that excessive alcohol use results annually in 140,000 deaths and $249 billion in economic costs. Treatment options for alcohol dependence include psychosocial treatment (including twelve-step facilitation programs) and medication (naltrexone, acamprosate). The former are effective but require continuous vigilance and frequently result in relapse. Existing medications to treat alcohol dependence have gained little traction because they are only partly effective and have serious side-effects, such as kidney damage.

Thus, there is a large, unmet need for an effective medical intervention that can change the underlying biological cause of alcohol dependence to augment behavioral interventions.

The present invention addresses this need.

### SUMMARY OF THE INVENTION

Experiments conducted during the course of developing embodiments for the present invention demonstrated that inhibiting alcohol-related NMDA receptor up-regulation as an effective treatment for alcohol dependence and effective technique for reducing alcohol consumption and binge drinking behavior. Such experiments further indicated rapastinel as an effective NMDA agonist capable of inhibiting alcohol-related NMDA receptor up-regulation.

Accordingly, the present invention relates to methods of treating or preventing alcohol dependence, enhancing the treatment of alcohol dependence, treating alcohol withdrawal, reducing alcohol consumption, alleviating one or more alcohol withdrawal symptoms in a subject, preventing or reducing the likelihood of alcohol dependence relapse in a subject treated for alcohol dependence, preventing alcohol-related N-methyl-D-aspartate receptor (NMDA) upregulation, preventing alcohol-related NMDA receptor activity, comprising administering a therapeutically effective amount of an NMDA receptor partial agonist (e.g., rapastinel) to the subject. The present invention further relates to compositions comprising an NMDA receptor partial agonist (e.g., rapastinel) for use with the aforementioned methods.

In certain embodiments, the present invention provides a composition comprising an NMDA partial agonist. In some embodiments, the NDMA partial agonist is selected from N-methyl-D-aspartic acid, 3,5-dibromo-L-phenylalanine, rapastinel, apimostinel, aminocyclopropanecarboxylic acid, cycloserine, HA-966, NYX-2925, and homoquinolinic acid. In some embodiments, the NDMA partial agonist is rapastinel.

In certain embodiments, the present invention provides a method for treating or preventing alcohol dependence in a subject, comprising administering a therapeutically effective amount of a composition comprising an NMDA receptor partial agonist (e.g., rapastinel) to the subject.

In certain embodiments, the present invention provides a method for reducing alcohol consumption in a subject, comprising administering a therapeutically effective amount of a composition comprising an NMDA receptor partial agonist (e.g., rapastinel) to the subject.

In certain embodiments, the present invention provides a method for preventing or inhibiting alcohol-related NMDA receptor upregulation in a subject, comprising administering a therapeutically effective amount of a composition comprising an NMDA receptor partial agonist (e.g., rapastinel) to the subject.

In certain embodiments, the present invention provides a method for preventing or inhibiting alcohol-related NMDA receptor activity in a subject, comprising administering a therapeutically effective amount of a composition comprising an NMDA receptor partial agonist (e.g., rapastinel) to the subject.

In certain embodiments, the present invention provides a method for enhancing the treatment of alcohol dependence in a subject, comprising administering a therapeutically effective amount of a composition comprising an NMDA receptor partial agonist (e.g., rapastinel) to the subject.

In certain embodiments, the present invention provides a method for treating or preventing or ameliorating withdrawal symptoms related to alcohol dependence in a subject, comprising administering a therapeutically effective amount of a composition comprising an NMDA receptor partial agonist (e.g., rapastinel) to the subject.

In certain embodiments, the present invention provides a method of claim 9, wherein the alcohol withdrawal symptoms include one or more of: insomnia, fatigue, tremor, anxiety, restlessness/agitation, nausea and vomiting, headache, excessive sweating, palpitations, anorexia, depression, craving for alcohol, alcoholic hallucinosis, seizures, and delirium tremens.

In certain embodiments, the present invention provides a method for preventing alcohol dependence relapse in a subject treated or being treated for alcohol dependence, comprising administering a therapeutically effective amount of a composition comprising an NMDA receptor partial agonist (e.g., rapastinel) to the subject.

In certain embodiments, the present invention provides a method for treating alcohol and opioid dependence in a subject suffering from dual dependence comprising, comprising administering a therapeutically effective amount of a composition comprising an NMDA receptor partial agonist (e.g., rapastinel) to the subject.

In certain embodiments, the present invention provides a method for enhancing the treatment of alcohol and opioid dependence in a subject suffering from dual dependence comprising, comprising administering a therapeutically effective amount comprising an NMDA receptor partial agonist (e.g., rapastinel) to the subject.

In certain embodiments, the present invention provides a method for treating alcohol and opioid withdrawal in a subject suffering from dual dependence, comprising administering a therapeutically effective amount comprising an NMDA receptor partial agonist (e.g., rapastinel) to the subject.

In certain embodiments, the present invention provides a method for preventing alcohol and opioid dependence relapse in a subject treated for alcohol and opioid dual dependence, comprising administering a therapeutically effective amount of a composition comprising an NMDA receptor partial agonist (e.g., rapastinel) to the subject.

In certain embodiments, the present invention provides a method for reducing the vulnerability of a subject to develop alcohol and opioid dual dependence, comprising administering a therapeutically effective amount of a composition comprising an NMDA receptor partial agonist (e.g., rapastinel) to the subject.

In certain embodiments, the present invention provides a method for reducing the vulnerability of a subject to experience binge drinking of alcohol, comprising administering a therapeutically effective amount of a composition comprising an NMDA receptor partial agonist (e.g., rapastinel) to the subject.

In certain embodiments, the present invention provides a method for reducing the vulnerability of a subject to experience binge drinking of alcohol and binge use of opioids, comprising administering a therapeutically effective amount of a composition comprising an NMDA receptor partial agonist (e.g., rapastinel) to the subject.

In certain embodiments, the present invention provides a method for regulating the amount of alcohol consumed by a subject during an occasion for purposes of avoiding binge drinking of alcohol, comprising administering a therapeutically effective amount of a composition comprising an NMDA receptor partial agonist (e.g., rapastinel) to the subject.

In certain embodiments, the present invention provides a method for regulating the amount of alcohol and opioids consumed by a subject during an occasion for purposes of avoiding binge drinking of alcohol, comprising administering a therapeutically effective amount of a composition comprising an NMDA receptor partial agonist (e.g., rapastinel) to the subject.

In certain embodiments, the present invention provides a method for regulating binge drinking of alcohol behavior in a subject, comprising administering a therapeutically effective amount of a composition of claim 1 to the subject.

In certain embodiments, the present invention provides a method for regulating binge drinking of alcohol behavior and binge use of opioids in a subject, comprising administering a therapeutically effective amount of a composition of claim 1 to the subject.

In certain embodiments, the present invention provides a method of regulating consumption of an alcoholic beverage, the method comprising administering a therapeutically effective amount of a composition of claim 1 to the subject.

In certain embodiments, the present invention provides a method of regulating consumption of an alcoholic beverage and opioids, the method comprising administering a therapeutically effective amount of a composition of claim 1 to the subject.

In any of such methods, the subject is human subject. In any of such embodiments, the subject is a human subject experiencing or at risk of experiencing alcohol-related NMDA receptor upregulation. In any of such embodiments, the subject is a human subject experiencing or at risk of experiencing alcohol dependence. In any of such embodiments, the subject is a human subject is abusing or at risk of abusing alcohol.

Additional embodiments will be apparent to persons skilled in the relevant art based on the teachings contained herein.

### Definitions

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “treating,” “therapy,” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

As used herein, the term “subject” and “patient” are used interchangeably and refer to both human and nonhuman animals. The term “nonhuman animals” includes all vertebrates, e.g., mammals and non-mammals, such as nonhuman primates, sheep, dog, cat, horse, cow, chickens, amphibians, reptiles, and the like. In some embodiments, the subject comprises a human suffering from an alcohol dependency. In certain embodiments, the subject comprises an adolescent human suffering from an alcohol dependency.

The term “effective amount” or “therapeutically effective amount” refers to an amount sufficient to effect beneficial or desirable biological and/or clinical results.

As used herein, the term “enhancing” refers to the increase and/or further improvement of a treatment.

As used herein, “alcohol withdrawal symptoms” include, but are not limited to, anxiety, shaky hands, headache, nausea, vomiting, insomnia, sweating, hallucinations, seizures, delusions, delirium tremens (DTs), confusion, racing heart, high blood pressure, fever, heavy sweating, and the like.

As used herein, “opioid withdrawal symptoms” include, but are not limited to, hyperalgesia, insomnia, anhedonia, dilated pupils, diarrhea, and cravings for opioids.

As used herein, “reducing the likelihood of alcohol dependence relapse” means decreasing the percent chance of relapsing, increasing the time to relapse, or both.

As used herein, “reducing the likelihood of opioid dependence relapse” means decreasing the percent chance of relapsing, increasing the time to relapse, or both.

A “pharmaceutically acceptable excipient,” “pharmaceutically acceptable carrier,” “a pharmaceutically acceptable diluent,” or “diagnostically acceptable excipient” includes but is not limited to, sterile distilled water, saline, phosphate buffered solutions, amino acid-based buffers, or bicarbonate buffered solutions. The excipient selected and the amount of excipient used will depend upon the mode of administration. It is also contemplated that the compositions of the invention or those used with the methods of the invention will contain certain pharmaceutically acceptable inert ingredients which are within the purview of one of skill in the art.

Unless otherwise defined, all technical terms used herein have the same meaning as commonly understood by one of ordinary skill in the art to which this disclosure belongs.

### BRIEF DESCRIPTION OF THE DRAWINGS

FIG. 1: Time course of exposure to alcohol under DID protocol and treatment with Rapastinel vs. saline.

FIG. 2A-2B: Rapastinel had no effect compared to saline control in alcohol consumption in mice that were alcohol naïve. Daily alcohol consumption (a) and total alcohol consumption (b) during 1 cycle of DID where mice underwent daily i.p. injection of 30 mg/kg Rapastinel or saline prior to presentation of alcohol.

FIG. 3A-3B: Individual mice have variable drinking behavior. Average consumption of alcohol by day during DID cycle over course of weeks 1-5 (a). Total average weekly ethanol consumption by individual animals with 3 noted to be greater than 90% (b).

FIG. 4A-4B: Rapastinel reduced alcohol consumption with and without removal of outliers. Total average weekly alcohol consumption over weeks 1-5 in comparison to week 6 with treatment with saline or rapastinel with (a) and without (b) inclusion of mice with individual consumption patterns >90%.

### DETAILED DESCRIPTION OF THE INVENTION

Experiments conducted during the course of developing embodiments for the present invention demonstrated that inhibition of alcohol-related NMDA receptor up-regulation as an effective treatment for alcohol dependence and effective technique for reducing alcohol consumption and binge drinking behavior. Such experiments further indicated rapastinel as an effective NMDA agonist capable of inhibiting alcohol-related NMDA receptor up-regulation.

Accordingly, he present invention relates to methods of treating or preventing alcohol dependence, enhancing the treatment of alcohol dependence, treating alcohol withdrawal, reducing alcohol consumption, alleviating one or more alcohol withdrawal symptoms in a subject, preventing or reducing the likelihood of alcohol dependence relapse in a subject treated for alcohol dependence, preventing alcohol-related NMDA receptor upregulation, preventing alcohol-related NMDA receptor activity, comprising administering a therapeutically effective amount of an NMDA receptor partial agonist (e.g., rapastinel) to the subject. The present invention further relates to compositions comprising an NMDA receptor partial agonist (e.g., rapastinel) for use with the aforementioned methods.

The NMDA receptor is found in nerve cells and is an ionotropic glutamate receptor. An NMDA receptor is activated when glutamate (or aspartate) and glycine (or D-serine) bind to it; binding of these co-agonists is required for efficient opening of the ion channel. NMDA partial agonists bind to the glutamate recognition site, the glycine recognition site, or to an allosteric site. Exemplary NMDA partial agonists include, but are not limited to, N-methyl-D-aspartic acid, 3,5-dibromo-L-phenylalanine, rapastinel, apimostinel, aminocyclopropanecarboxylic acid, cycloserine, HA-966, NYX-2925, and homoquinolinic acid.

In certain embodiments, the NMDA partial agonist used in the methods disclosed herein

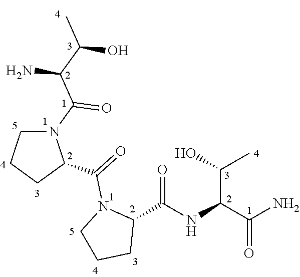

is rapastinel: L-threonyl-L-prolyl-L-prolyl-L-threoninamide. Rapastinel, an allosteric modulator of the glycine site of the NMDA receptor complex, has shown potential clinical efficacy against depression without the psychiatric side effects, abuse potential, or reinforcement of ketamine and other glutamate-targeted drugs. Unlike ketamine, rapastinel has not been shown to produce any negative side effects during treatment in animal models (see, Moskal, et al., Curr Neuropharmacol. 2017 January; 15 (1): 47-56.). Rapastinel has been described as an agent that “stabilizes” NMDA receptor function, preventing excessive activation, but permitting activation when it is deficient. Rapastinel is a safe, fast-acting, long-lasting therapeutic free of psychoactive side effects like those seen with the similar therapy ketamine.

As disclosed in the experiments described herein, rapastinel has been demonstrated to effectively inhibit alcohol-related NMDA receptor up-regulation, thereby serving as an effective agent for treating alcohol dependence and as an effective agent for reducing alcohol consumption.

In certain embodiments, the present invention provides methods for treating or preventing alcohol dependence in a subject, comprising administering a therapeutically effective amount of an NMDA partial agonist to the subject. In certain embodiments, the present invention provides methods for treating or preventing alcohol dependence in a subject, comprising administering a therapeutically effective amount of rapastinel to the subject.

In certain embodiments, the present invention provides methods for reducing alcohol consumption in a subject, comprising administering a therapeutically effective amount of an NMDA partial agonist to the subject. In certain embodiments, the present invention provides methods for reducing alcohol consumption in a subject, comprising administering a therapeutically effective amount of rapastinel to the subject.

In certain embodiments, the present invention provides methods for preventing or inhibiting alcohol-related NMDA receptor upregulation in a subject, comprising administering a therapeutically effective amount of an NMDA partial agonist to the subject. In certain embodiments, the present invention provides methods for preventing or inhibiting alcohol-related NMDA receptor upregulation in a subject, comprising administering a therapeutically effective amount of rapastinel to the subject.

In certain embodiments, the present invention provides methods for preventing or inhibiting alcohol-related NMDA receptor activity in a subject, comprising administering a therapeutically effective amount of an NMDA partial agonist to the subject. In certain embodiments, the present invention provides methods for preventing or inhibiting alcohol-related NMDA receptor activity in a subject, comprising administering a therapeutically effective amount of rapastinel to the subject.

In certain embodiments, the present invention provides methods for enhancing the treatment of alcohol dependence in a subject, comprising administering a therapeutically effective amount of an NMDA partial agonist to the subject. In certain embodiments, the present invention provides methods for enhancing the treatment of alcohol dependence in a subject, comprising administering a therapeutically effective amount of rapastinel to the subject.

In certain embodiments, the present invention provides methods for treating or preventing or ameliorating withdrawal symptoms related to alcohol dependence in a subject, comprising administering a therapeutically effective amount of an NMDA partial agonist to the subject. In certain embodiments, the present invention provides methods for treating or preventing or ameliorating withdrawal symptoms related to alcohol dependence in a subject, comprising administering a therapeutically effective amount of rapastinel to the subject. Such methods are not limited to specific alcohol withdrawal symptoms. In some embodiments, the alcohol withdrawal symptoms include, but are not limited to, insomnia, fatigue, tremor, anxiety, restlessness/agitation, nausea and vomiting, headache, excessive sweating, palpitations, anorexia, depression, craving for alcohol, alcoholic hallucinosis, seizures, and delirium tremens.

In certain embodiments, the present invention provides methods for preventing alcohol dependence relapse in a subject treated or being treated for alcohol dependence, comprising administering a therapeutically effective amount of an NMDA partial agonist to the subject. In certain embodiments, the present invention provides methods for preventing alcohol dependence relapse in a subject treated or being treated for alcohol dependence, comprising administering a therapeutically effective amount of rapastinel to the subject. In some embodiments, the subject has been treated or is being treated for alcohol dependence with a psychosocial treatment (including twelve-step facilitation programs) and/or a medication treatment (e.g., naltrexone, acamprosate).

In certain embodiments, the present disclosure provides a method of treating alcohol and opioid dependence in a subject suffering from dual dependence comprising, comprising administering a therapeutically effective amount of an NMDA partial agonist to the subject. In certain embodiments, the present disclosure provides a method of treating alcohol and opioid dependence in a subject suffering from dual dependence comprising, comprising administering a therapeutically effective amount of rapastinel to the subject.

In certain embodiments, the present disclosure provides a method of enhancing the treatment of alcohol and opioid dependence in in a subject suffering from dual dependence comprising, comprising administering a therapeutically effective amount of an NMDA partial agonist to the subject. In certain embodiments, the present disclosure provides a method of enhancing the treatment of alcohol and opioid dependence in in a subject suffering from dual dependence comprising, comprising administering a therapeutically effective amount of rapastinel to the subject.

In certain embodiments, the present disclosure provides a method of treating alcohol and opioid withdrawal in a subject suffering from dual dependence, comprising administering a therapeutically effective amount of an NMDA partial agonist to the subject. In certain embodiments, the present disclosure provides a method of treating alcohol and opioid withdrawal in a subject suffering from dual dependence, comprising administering a therapeutically effective amount of rapastinel to the subject.

In certain embodiments, the present disclosure provides a method of preventing alcohol and opioid dependence relapse in a subject treated for alcohol and opioid dual dependence, comprising administering a therapeutically effective amount of an NMDA partial agonist to the subject. In certain embodiments, the present disclosure provides a method of preventing alcohol and opioid dependence relapse in a subject treated for alcohol and opioid dual dependence, comprising administering a therapeutically effective amount of rapastinel to the subject.

In certain embodiments, the present disclosure provides a method of reducing the vulnerability of a subject to develop alcohol and opioid dual dependence, comprising administering a therapeutically effective amount of an NMDA partial agonist to the subject. In certain embodiments, the present disclosure provides a method of reducing the vulnerability of a subject to develop alcohol and opioid dual dependence, comprising administering a therapeutically effective amount of rapastinel to the subject.

In certain embodiments, the present disclosure provides a method for reducing the vulnerability of a subject to experience binge drinking of alcohol, comprising administering a therapeutically effective amount of an NMDA partial agonist to the subject.

In the context of embodiments of the present invention, regulating binge drinking relates to controlling the excessive behavior of indulgence in drinking alcoholic beverages. For example, regulating binge drinking may involve reducing the amount of alcohol consumed in a drinking session and/or the number of drinking sessions in a week, per the embodiments described hereinabove.

Regulating binge drinking provides restraining or moderating the compulsive uncontrollable, behavior associated with binge drinking thereby reducing the harmful consequences associated with excessive alcohol consumption, while not impairing the psychological and social pleasures associated with the drinking.

The phrase “binge drinking” or “binge drinking disorder” as used herein is determined according national or international definitions as defined by regulation authorities, for example, in line with the definition of binge drinking used by the National Health Service (NHS) and the National Office of Statistics in the United Kingdom (UK), and corresponding services, offices and/or authorities in other countries. The definition of binge drinking used by NHS and the National Office of Statistics describes it as drinking more than double the lower risk guidelines for alcohol in one drinking session, wherein the guidelines advise that people should not regularly drink more than the lower risk guidelines of 3-4 units of alcohol for men (equivalent to a pint and a half of 4% beer (about 852 ml)) and 2-3 units of alcohol for women (equivalent to a 175 ml glass of wine). “Regularly” means drinking every day or most days of the week. “One unit of alcohol” as defined herein and acceptable in the art is 10 ml of pure alcohol. It takes an average adult around an hour to process this so that there's none left in the bloodstream, although this varies from person to person.

In some embodiments, binge drinking for men, therefore, is regularly drinking more than 8 units of alcohol—or about three pints of strong beer. For women, it's regularly drinking more than 6 units of alcohol, equivalent to 2¼ pints of strong beer or two large glasses of wine.

In an aspect of some embodiments of the present invention, there is provided a method of regulating consumption of an alcoholic beverage in a subject in need thereof, the method being effected by administering to the subject a therapeutically effective amount of a composition comprising an NMDA receptor partial agonist (e.g., rapastinel). In some embodiments, the consumption of alcohol beverages is excessive and uncontrolled as in binge drinking. In some embodiments, the subject is determined as having a binge drinking disorder, as defined herein. In some of these embodiments, the method provided herein is for regulating binge drinking or treating a binge drinking disorder in a subject in need thereof.

In some embodiments, the method provided herein is for reducing alcohol consumption in a non-binger subject who wishes to control his alcohol consumption, for example, under certain circumstances (for example, during a specific event or time point).

The term “binge drinking regulation” in a broad interpretation refers to controlling the excessive, uncontrolled consumption of alcoholic beverages. Regulating binge drinking relates to reducing the amount of alcohol consumed in a drinking session and/or reducing the number of drinking sessions.

In the context of embodiments of the present invention, binge drinking regulation relates to imparting a feeling of satisfaction, satiety or contentedness which discourages binge drinking. Binge drinking regulation as practiced in embodiments of the present invention effects a true harm reduction utility by discouraging binge drinking in a way that is easy to implement (by drinking) and non harmful (pending toxicological validation) to the drinker.

Such methods are not limited to a particular type or kind of NMDA partial agonist. In some embodiments, the NMDA partial agonist is selected from N-methyl-D-aspartic acid, 3,5-dibromo-L-phenylalanine, rapastinel, apimostinel, aminocyclopropanecarboxylic acid, cycloserine, HA-966, NYX-2925, and homoquinolinic acid.

Such methods are not limited to a particular type of subject. In some embodiments, the subject is human subject. In some embodiments, the subject is a human subject experiencing or at risk of experiencing alcohol-related NMDA receptor upregulation. In some embodiments, the subject is a human subject experiencing or at risk of experiencing alcohol dependence. In some embodiments, the subject is a human subject is abusing or at risk of abusing alcohol.

Compositions within the scope of this disclosure include all compositions wherein an NMDA receptor partial agonist (e.g., rapastinel) is contained in an amount which is effective to achieve its intended purpose. While individual needs vary, determination of optimal ranges of effective amounts of each component is within the skill of the art. Typically, an NMDA receptor partial agonist (e.g., rapastinel) may be administered to subjects, e.g., human subjects suffering or risk of suffering from alcohol dependence, orally at a dose of 0.0025 to 100 mg/kg, or an equivalent amount of the pharmaceutically acceptable salt thereof, per day of the body weight of the mammal being treated for disorders responsive to induction of apoptosis. In one embodiment, about 0.01 to about 25 mg/kg of ESK981 is orally administered to treat, ameliorate, or prevent alcohol dependence and/or alcohol-related NMDA receptor upregulation.

For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of an NMDA receptor partial agonist (e.g., rapastinel). The unit dose may be administered one or more times daily as one or more tablets or capsules each containing from about 0.1 to about 10 mg, conveniently about 0.25 to 50 mg of an NMDA receptor partial agonist (e.g., rapastinel).

In a topical formulation, an NMDA receptor partial agonist (e.g., rapastinel) may be present at a concentration of about 0.01 to 100 mg per gram of carrier. In a one embodiment, an NMDA receptor partial agonist (e.g., rapastinel) is present at a concentration of about 0.07-1.0 mg/ml, for example, about 0.1-0.5 mg/ml, and in one embodiment, about 0.4 mg/ml.

In addition to administering an NMDA receptor partial agonist (e.g., rapastinel) as a raw chemical, the NMDA receptor partial agonist (e.g., rapastinel) may be administered as part of a pharmaceutical formulation containing suitable pharmaceutically acceptable carriers comprising excipients and auxiliaries which facilitate processing of an NMDA receptor partial agonist (e.g., rapastinel) into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of ESK981, together with the excipient.

The pharmaceutical compositions comprising an NMDA receptor partial agonist (e.g., rapastinel) may be administered to any subject which may experience the beneficial effects of an NMDA receptor partial agonist (e.g., rapastinel). Foremost among such subjects are mammals, e.g., humans, although the disclosure is not intended to be so limited. Other subjects include veterinary animals (cows, sheep, pigs, horses, dogs, cats and the like).

An NMDA receptor partial agonist (e.g., rapastinel) and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present disclosure are manufactured in a manner which is itself known, for example, by means of conventional mixing, granulating, dragee-making, dissolving, or lyophilizing processes. Thus, pharmaceutical preparations for oral use can be obtained by combining an NMDA receptor partial agonist (e.g., rapastinel) with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this disclosure are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C12). The carriers may be those in which the active ingredient is soluble. Emulsifiers, stabilizers, humectants and antioxidants may also be included as well as agents imparting color or fragrance, if desired.

Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

The present disclosure further provides kits comprising the compositions provided herein and for carrying out the subject methods as provided herein. For example, in one embodiment, a subject kit may comprise, consist of, or consist essentially of an NMDA partial agonist and/or pharmaceutical compositions as provided herein.

In other embodiments, a kit may further include other components. Such components may be provided individually or in combinations, and may provide in any suitable container such as a vial, a bottle, or a tube. Examples of such components include, but are not limited to, one or more additional reagents, such as one or more dilution buffers; one or more reconstitution solutions; one or more wash buffers; one or more storage buffers, one or more control reagents and the like. Components (e.g., reagents) may also be provided in a form that is usable in a particular assay, or in a form that requires addition of one or more other components before use (e.g. in concentrate or lyophilized form). Suitable buffers include, but are not limited to, phosphate buffered saline, sodium carbonate buffer, sodium bicarbonate buffer, borate buffer, Tris buffer, MOPS buffer, HEPES buffer, and combinations thereof.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

The following example is provided in order to demonstrate and further illustrate certain preferred embodiments and aspects of the present invention and are not to be construed as limiting the scope thereof. Use of pronouns such as, “we”, “our,” and “I” refer to the inventive entity.

### Example 1

Alcohol use disorder is a disabling condition with current medication therapies often ineffective in reducing alcohol consumption and preventing relapse. Chronic ethanol exposure has been linked to altered glutamatergic transmission that may contribute to maladaptive behaviors in alcohol use disorder (see, Alasmari F, Goodwani S, McCullumsmith R E, Sari Y (2018): Role of glutamatergic system and mesocorticolimbic circuits in alcohol dependence. Prog Neurobiol. 171: 32-49). Rapastinel is an NMDA-receptor positive allosteric modulator that has previously been shown to be effective in reducing cravings for opiates in preclinical rodent models (see, Thiele T E, Crabbe J C, Boehm S L, 2nd (2014): “Drinking in the Dark” (DID): a simple mouse model of binge-like alcohol intake. Curr Protoc Neurosci. 68: 9 49 41-49).

Experiments were conducted to demonstrate the effect of Rapastinel, an NMDA receptor modulator, in a mouse model of binge drinking behavior. To measure binge drinking behavior, experiments were conducted that used the Drinking in the Dark (DID) model as previously published (see, Thiele T E, Crabbe J C, Boehm S L, 2nd (2014): “Drinking in the Dark” (DID): a simple mouse model of binge-like alcohol intake. Curr Protoc Neurosci. 68: 9 49 41-49 49 12). Adult male C57/B16J mice on reverse light-dark cycle had water replaced with alcohol three hours into the dark cycle. On days 1-3 of each week (Mon-Wed), mice received 20% alcohol for 2 hrs and on day 4 (Thur) received for 4 hours. Mice did not receive alcohol on days 5-7 of each cycle (Fri-Sun).

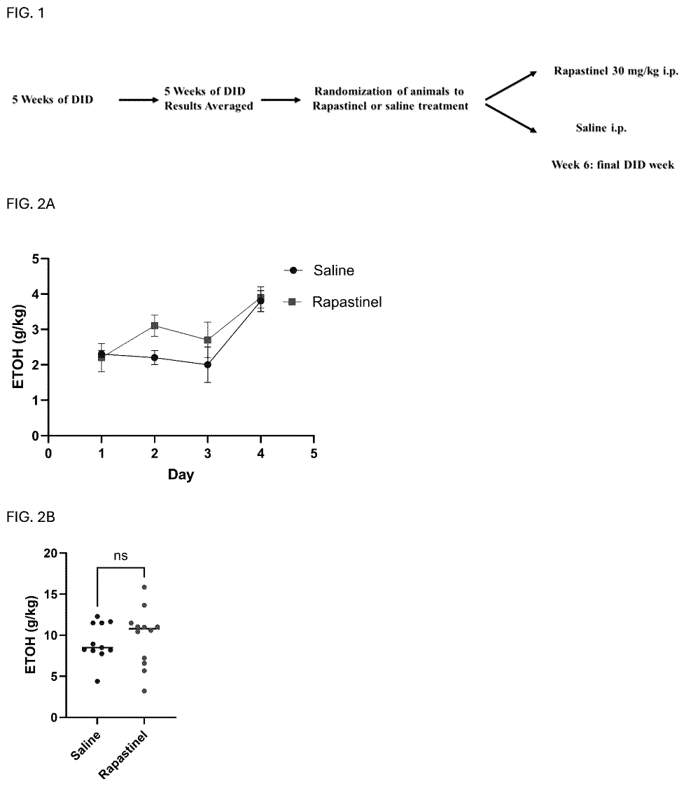

Mice were treated with Rapastinel at dose of 30 mg/kg or saline via intraperitoneal injection (i.p.) 30 minutes prior to alcohol exposure on days 1-4 of cycle either during first cycle of DID or after first undergoing DID for 5 weeks prior to treatment on week 6 (see, FIG. 1).

Rapastinel was shown to have no effect compared to saline control in alcohol consumption in mice that were alcohol naïve. Daily alcohol consumption (a) and total alcohol consumption (b) during 1 cycle of DID were mice underwent daily i.p. injection of 30 mg/kg Rapastinel or saline prior to presentation of alcohol (see, FIG. 2).

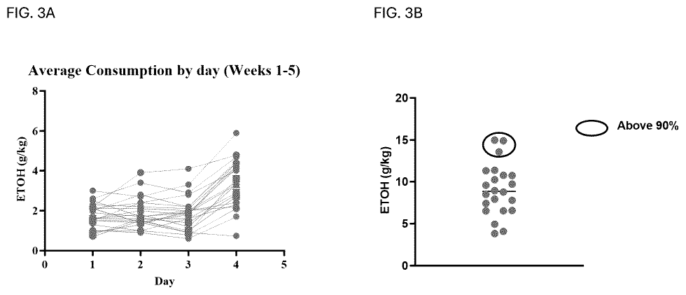

Individual mice were shown to have variable drinking behavior. Average consumption of alcohol by day during DID cycle over course of weeks 1-5 (a). Total average weekly ethanol consumption by individual animals with 3 noted to be greater than 90% (b) (see, FIG. 3).

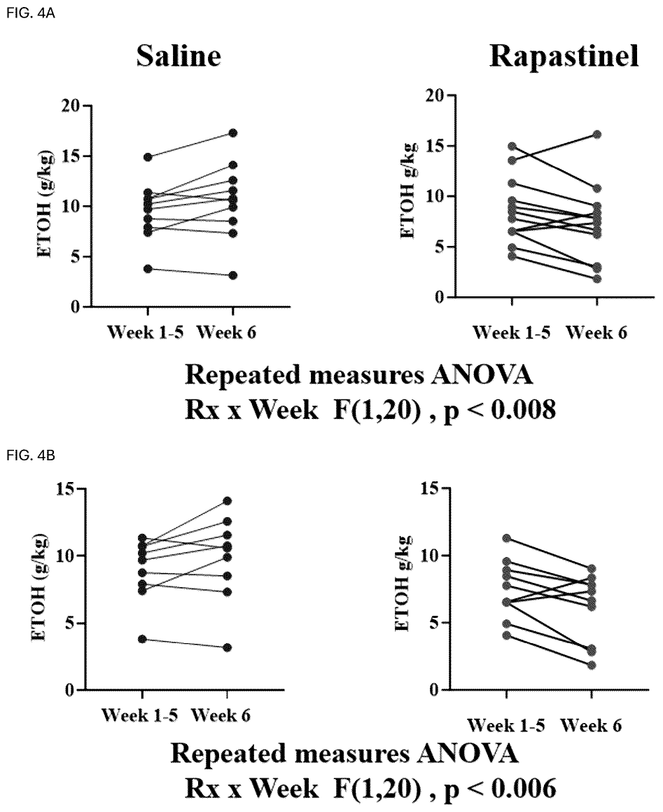

Rapastinel was shown to reduce reduced alcohol consumption with and without removal of outliers. Total average weekly alcohol consumption over weeks 1-5 in comparison to week 6 with treatment with saline or rapastinel with (a) and without (b) inclusion of mice with individual consumption patterns >90% (see, FIG. 4).

These results indicate that Rapastinel can reduce binge drinking behaviors in mice and may provide a novel therapeutic approach in the treatment of alcohol use disorder.

### Equivalents

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

The entire disclosure of each of the patent documents and scientific articles referred to herein is incorporated by reference for all purposes.

1. A composition comprising an NMDA partial agonist.

2. The composition of claim 1, wherein the NDMA partial agonist is selected from N-methyl-D-aspartic acid, 3,5-dibromo-L-phenylalanine, rapastinel, apimostinel, aminocyclopropanecarboxylic acid, cycloserine, HA-966, NYX-2925, and homoquinolinic acid.

3. The composition of claim 1, wherein the NDMA partial agonist is rapastinel.

4. A method for one or more of the following:
treating or preventing alcohol dependence in a subject,
reducing alcohol consumption in a subject,
preventing or inhibiting alcohol-related NMDA receptor upregulation in a subject,
preventing or inhibiting alcohol-related NMDA receptor activity in a subject,
enhancing the treatment of alcohol dependence in a subject,
treating or preventing or ameliorating withdrawal symptoms related to alcohol dependence in a subject,
preventing alcohol dependence relapse in a subject treated or being treated for alcohol dependence,
treating alcohol and opioid dependence in a subject suffering from dual dependence,
treating alcohol and opioid withdrawal in a subject suffering from dual dependence,
preventing alcohol and opioid dependence relapse in a subject treated for alcohol and opioid dual dependence,
reducing the vulnerability of a subject to develop alcohol and opioid dual dependence,
reducing the vulnerability of a subject to experience binge drinking of alcohol regulating binge drinking of alcohol behavior in a subject,
regulating consumption of an alcoholic beverage,
comprising administering a therapeutically effective amount of a composition of claim 1 to the subject.

5-19. (canceled)

20. The method of claim 4, wherein the subject is human subject.

21. The method of claim 4, wherein the subject is a human subject experiencing or at risk of experiencing alcohol-related NMDA receptor upregulation.

22. The method of claim 4, wherein the subject is a human subject experiencing or at risk of experiencing alcohol dependence.

22. The method of claim 4, wherein the subject is a human subject is abusing or at risk of abusing alcohol.
